(19) United States
(12) Patent Application Publication
(10) Pub. No.: US 2019/0134123 A1
(43) Pub. Date: May 9, 2019

(54) NOVEL SYSTEM AND METHOD FOR MICROBIOME PROFILING AND MODULATION BY MEANS OF CANNABIS ADMINISTRATION

(71) Applicant: Cannabics Pharmaceuticals Inc., Bethesda, MD (US)

(72) Inventors: Eyal BALLAN, Ramat Hasharon (IL); Moran GRINBERG, Holon (IL)

(21) Appl. No.: 16/182,726

(22) Filed: Nov. 7, 2018

Related U.S. Application Data
(60) Provisional application No. 62/582,966, filed on Nov. 8, 2017.

(51) Int. Cl. A61K 36/185; A61K 31/05; A61K 31/352; G01N 33/50

(52) U.S. Cl. CPC A61K 36/185; G01N 33/5011; A61K 31/352; A61K 31/05

(57) ABSTRACT

Novel means and methods for personalized diagnosing and profiling health status in mammalian subjects, as well as optimization of pharmacological treatments for patients, based on profiling and modulating of patient-derived microbiota. Combined method and system for selecting the optimal cannabinoid-based treatment modality for a range of cancerous and infective diseases and disorders by modulating said subject microbiome.

### FIELD OF THE INVENTION

The present disclosure relates to novel means and methods for personalized diagnosing and profiling health status in mammalian subjects, as well as optimization of pharmacological treatments for patients, based on profiling and modulating of patient-derived microbiota. More particularly, the current invention pertains to a combined method and system for selecting the optimal cannabinoid-based treatment modality for a range of cancerous and infective diseases and disorders by modulating said subject microbiome.

### BACKGROUND OF THE INVENTION

### Cancer Detection and Treatment

Cancer or malignant neoplasm is a genetic disorder that results from genetic or epigenetic alterations in the somatic cells. Tumorigenesis in humans is a multistep process which involves various genetic or epigenetic changes which ultimately drive the malignant transformation of the normal cells.

Cancer is clearly the most deadly disease in the developed world as one in three people develop cancer during their lifetime. The cure for cancer is an unmet need, since most of the existing treatments are not effective enough to provide full protection from this disease. Furthermore, several limitations which are associated with cancer diagnosis make it difficult to treat: The non-specific nature of cancer symptoms makes diagnosis difficult. In certain cases the patient remains asymptotic. So these early signs and symptoms of cancer are often neglected by the patient which provides the opportunity for the cancer to spread without any medical intervention. By the time the patient seeks medical help, it may be out of reach of available clinical treatment. The diagnosis difficulties of cancers can be shown for example in esophageal cancer. Esophageal cancer is one of the most lethal cancers and it is difficult to treat. Unfortunately, early detection of this type of cancer is very difficult simply because in the early phase of this cancer smaller tumors often cause few or no symptoms. However undetected esophageal cancer can spread into various parts of the body including the stomach, lungs, liver and lymph nodes. In the late metastasized stage the tumor is incurable and most of the treatment of late stage only focuses on extending life and relieving the symptoms.

The unavailability of effective biomarkers for cancer diagnosis and prognosis is another hindrance for cancer treatment. Biomarkers are not only important for diagnostic purposes but can also be of pronounced prognostic value. With the identification of the right biomarker the cancer progression and effect of chemotherapeutic drugs can be evaluated in great detail. But unfortunately the hunt is still on to identify reliable biomarkers for different cancers. Further research, standardization and validation of this approach is needed.

Another obstacle for cancer treatment is the limitations of conventional chemotherapeutic agents. The existing chemotherapeutic drugs are toxic to all cells including cancer and normal cells. So the administration of these toxic agents kill the rapidly proliferating cancer cells as well as the normal cells which may lead to some serious side effects and may sometimes cause the death of patients. Untargeted radiotherapy suffers from a similar lack of specificity.

Thus all above-mentioned reasons for the difficulties in cancer treatment are still an unmet need for early detection and reduction of the incidence of different types of cancer.

### Infectious Diseases

Chronic infections are a major concern for public health. Although many infectious diseases are acute, most deaths and debility attributed to infections, are due to chronic parasitic, fungal, mycobacterial, and viral infections. Chronic infections are globally dominant diseases where treatment is often showing an increasing antimicrobial resistance. New therapeutic strategies are therefore desirable.

The mechanisms of chronic infectious diseases remain poorly understood, and optimal methods for their treatment are still to be found.

### Infectious Diseases: Gonorrhea

Neisseria gonorrhoeae (the gonococcus) is a Gram-negative diplococcus, an obligate human pathogen, and the etiologic agent of the sexually transmitted disease, gonorrhea. Infections lead to limited immunity, therefore individuals can become repeatedly infected The gonococcus infects a diverse array of mucosal surfaces, some of which include the urethra, the endocervix, the pharynx, conjunctiva and the rectum. In 2013, the Centers for Disease Control and Prevention (CDC) reported that there were 333,004 new cases of gonorrhea in the United States, with an incidence of 106.1 cases per 100,000 population. Worldwide, 106.1 million people are infected by N. gonorrhoeae annually. Gonorrhea is generally a non-complicated mucosal infection with a pustular discharge. More severe sequellae include salpingitis and pelvic inflammatory disease which may lead to sterility and/or ectopic pregnancy. Occasionally, the organism can disseminate as a bloodstream infection. If left untreated, these more serious complications can result in occasionally death. Approximately 3% of women presenting with a urogenital infection develop the most severe forms of the disease. When the bacteria do cross the endothelium, they can spread to other locations in the body.

Treatment and curability: Gonorrhea is susceptible to an array of antibiotics. Antibiotic resistance is becoming a major problem and there are fears that the gonococcus will become the next “superbug” as the antibiotic arsenal diminishes.

### Fungal Infections

Invasive, life-threatening fungal infections are an important cause of morbidity and mortality, particularly for patients with compromised immune function. The number of therapeutic options for the treatment of invasive fungal infections is quite limited. Indeed, only three classes of molecules are currently used in clinical practice and only one new class of antifungal drugs has been developed in the last 30 years.

Over the past 30 years, the importance of antifungal drugs to the practice of modern medicine has increased dramatically. Because the vast majority of life-threatening fungal infections affect people with altered immune function, the increased incidence of invasive fungal infections can be correlated with an expansion in the number of people living with conditions or treatments that affect immune function, examples of which include HIV/AIDS, primary immune deficiency, cancer chemotherapy, hematologic and solid organ transplantation, prematurity, and immune-modulatory medications.

However, even with these newest therapies, the clinical outcomes for most invasive fungal infections are far from ideal. Indeed, infections caused by species of molds for which there is no reliable medical therapy are emerging as are strains of the more common organisms such as Candida albicans and Candida glabrata that are resistant to currently used drugs. Furthermore, Candida is a serious life-threatening pathogen, particularly in immunocompromised patients. Candida infections are considered as a major cause of morbidity and mortality in a broad range of immunocompromised patients. Candida infections are common in hospitalized patients and elderly people. The difficulty to eradicate Candida infections is owing to its unique switch between yeast and hyphae forms and more likely to biofilm formations that render resistance to antifungal therapy (see in the aforementioned paragraph). Antifungal treatment modality is therefore still an unmet need.

### Fungal Infections—Candida

Candida is a fungal pathogen, which is mostly known to cause high rate of mycotic infection to human worldwide. Candida is known to cause mucosal and deep tissue infections. Candida infects mucosal tissues including mouth, esophagus, gut, and vagina. Vaginal candidiasis continues to be a world health problem to women. Candidal infections are common in hospitalized patients and elderly people, and are difficult to control. About 50% of adults have Candida yeasts in their mouth and it is responsible for superficial easily treated infections. However, candidal infections can spread through the body and become life threatening, in particular with immunocompromised patients.

Candidiasis represents a major cause of death. Candida can switch between two major forms, yeast and hyphae forms. The switch from yeast to hyphae is considered a major infectious agent of Candida. In addition, Candida spp. produces biofilms on synthetic materials, which facilitates adhesion of the organisms to devices and renders the organism relatively resistant to antifungal therapy. Catheter-associated Candida biofilms can lead to bloodstream infections. Candida-infected catheters, in particular those associated with microbial biofilms, can represent 90% of infections among hospital-admitted patients and hence considered as a major cause of death. Several synthetic drugs are established in the treatment regimens of candidal infections, however drug resistance is developed.

The formation of biofilms in Candida and the transition from planktonic to sessile form are mainly associated with highly resistant phenotype. Other mechanisms of resistance include the expression of resistance genes, particularly those encoding efflux pumps, and the presence of persisted cells. Major synthetic drugs that develop candidal resistance include 5-flucytosin, amphotericin B, azoles, and echinocandins.

### SUMMARY OF THE INVENTION

It is thus one object of the present invention to disclose a method useful for personalized time determined profiling and modulating a microbiome of a mammalian subject, wherein said steps comprise:
a. identifying predetermined characteristics of said mammalian subject microbiota;
b. contacting in vitro a sample of said microbiota with a plurality of cannabinoid compounds and recording data;
c. selecting a cannabinoid composition comprising at least one cannabinoid compound determined to have modulated said microbiota in vitro and administering it to said mammalian subject;
d. monitoring response of said mammalian subject to said administration and correlating said response with said in vitro data
e. correlating said in vitro data with said response of said mammalian subject first cycle and optionally selecting a second cycle of said personalized profiling and modulation for said mammalian subject.

It is another object of the present invention to disclose the method mentioned above, wherein mammalian subject said is a human patient. It is another object of the present invention to disclose the method mentioned above, wherein said microbiota comprising at least one of bacteria, archaea, fungi, protists, viruses, and any combination thereof.

It is another object of the present invention to disclose the method mentioned above, wherein said method additionally comprising steps of identifying characteristics of circulating tumor cells (CTCs) of said mammalian subject.

It is another object of the present invention to disclose the method mentioned above, wherein said method additionally comprising steps of identifying characteristics of cancerous tissue of said mammalian subject.

It is another object of the present invention to disclose the method mentioned above, wherein said identifying characteristics of said mammalian subject microbiota is selected from the group of means consisting counting live cells, counting dead cells, counting apoptotic cells, measuring levels of RNA, measuring levels of DNA and any combination thereof.

It is another object of the present invention to disclose the method mentioned above, wherein said human patient selected from a group of patients not diagnosed with cancer, patients not diagnosed with microbial infections, patients diagnosed with cancer, patients diagnosed with cancer resistant to conventional chemotherapies, patients diagnosed with microbial infections and patients diagnosed with microbial infections resistant to conventional antibiotics and any combination thereof.

It is another object of the present invention to disclose the method mentioned above, wherein microbial infections comprising at least one of bacterial infections, fungal infections, viral diseases, parasite diseases, prion diseases and any combination thereof.

It is another object of the present invention to disclose the method mentioned above, wherein said microbial infection is Gonorrhea.

It is another object of the present invention to disclose the method mentioned above, wherein said microbial infection is Candida.

It is another object of the present invention to disclose the method mentioned above, wherein said response is selected of a group comprising: cardiac monitoring, hemodynamic monitoring, respiratory monitoring, neurological monitoring, blood glucose monitoring, childbirth monitoring, body temperature monitoring, vital parameters, local response monitoring and any combination thereof.

It is another object of the present invention to disclose the method mentioned above, wherein cancer is selected from gastric cancer, esophageal cancer, vaginal cancer, colorectal cancer, hepatobiliary cancer, pancreatic cancer, lung cancer, stomach cancer, head and neck cancer, breast cancer, lymphoma, cutaneous B-cells non-Hodgkin lymphoma, MALT lymphomas, Hodgkin lymphoma, colorectal adenoma and any combination thereof.

It is another object of the present invention to disclose the method mentioned above, wherein said cannabinoid is selected from the group consisting of cannabinoid-type, cannabinoid derivative, cannabis extract or fraction thereof, non-cannabinoid-type constituent, product, compound, molecule or substance and any combination thereof.

It is another object of the present invention to disclose the method mentioned above, wherein said cannabinoid compound is extracted from cannabis; said cannabis is selected from a group consisting of: Cannabis sativa, Cannabis indica, Cannabis ruderalis, and any combination thereof.

It is another object of the present invention to disclose the method mentioned above, wherein said cannabinoid compounds are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

It is another object of the present invention to disclose the method mentioned above, wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

It is another object of the present invention to disclose the method mentioned above, wherein the cannabidiol (CBD) or a derivative thereof is selected from the group consisting of CBD, CBDV, CBDA and any combination thereof.

It is another object of the present invention to disclose the method mentioned above, wherein said selecting of cannabinoid composition comprising at least one cannabinoid compound determined to have modulated said microbiota, adjunct to conventional chemotherapy; or said cannabinoid compound as mono-therapy.

It is another object of the present invention to disclose the method mentioned above, wherein said conventional chemotherapy is selected from the group consisting of chemotherapy, surgery, radiotherapy, hormonotherapy, and/or immunotherapy.

It is another object of the present invention to disclose the method mentioned above, wherein said selecting of cannabinoid composition comprising at least one cannabinoid compound determined to have modulated said microbiota adjunct to conventional antibiotics; therapy or said cannabinoid compound as mono-therapy.

It is another object of the present invention to disclose the method mentioned above, wherein said response is selected from the group consisting of: cancer markers level, tumor size monitoring, metastasis monitoring, survival, quality of life measured according to one or more scales, and any combination thereof.

It is another object of the present invention to disclose the method mentioned above, wherein said response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, inhibited microbial infection, inhibited microbial growth, improved mammalian subject survival, or any combination thereof.

It is another object of the present invention to disclose the method mentioned above, repeating the recording data on the outcome of in vitro contacting of said microbiota at a plurality of time points, determining whether the subject is responsive; and recommending the administration of the selected personalized therapy be continued if the subject is responsive, or to be discontinued is the subject is non responsive, i.e. resistant.

It is another object of the present invention to disclose the method mentioned above, wherein resistance to a drug is detected when there is uninhibited microbial infection, uninhibited microbial growth uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

It is another object of the present invention to disclose the method mentioned above, wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

It is another object of the present invention to disclose the method mentioned above, wherein the recording data on the outcome of in vitro contacting comprises steps selected from a group of isolating, enumerating, counting, purifying, sequencing, sensitizing, with a plurality of cannabinoid compounds and any combination thereof.

It is another object of the present invention to disclose the method mentioned above, wherein said recording data on the outcome of in vitro contacting is selected from the group of means consisting of: optic, luminescent, fluorescent, immunological, cell count, radioactive, non-radioactive isotopic, electrical and any combination thereof.

It is another object of the present invention to disclose the method mentioned above, wherein said method further comprising recording data of said in vitro contacting microbiota with a plurality of cannabinoid compounds at n time points, wherein n is an integer equal or higher than 2, comprising of at least one time point before start of said personalized modulation and at least a second time point at a time during said first cycle; and correlating said in vitro data with said therapeutic response of said first cycle and selecting a second cycle of said personalized modulation for said mammalian subject.

It is another object of the present invention to disclose the method mentioned above, wherein said method is additionally useful for early diagnosing of cancerous states.

It is another object of the present invention to disclose the method mentioned above, wherein said method is additionally useful for early diagnosing of microbial infections.

It is thus one object of the present invention to disclose a system useful for a personalized time-determined profiling and modulating a microbiome of mammalian subject, wherein the system comprises:
a. in vitro identifier module configured to identify pre-determined characteristics of said mammalian subject microbiota;
b. in vitro contact module configured to contact a sample of said microbiota with a plurality of cannabinoid compounds;
c. a recorder module for recording data on the outcome of said in vitro contacting
d. a module for selecting a cannabinoid composition comprising at least one cannabinoid compound determined to have modulated said microbiota for said mammalian subject and for administrating to said mammalian subject;
e. a module for monitoring response of said mammalian subject to said administration;
f. a module for correlating said in vitro data with said response of said mammalian subject first cycle and optionally selecting a second cycle of said personalized profiling and modulation of said mammalian subject.
g. a module for processing said detected signals with said therapeutic response of said first cycle and selecting a second cycle of clinical personalized therapy for said mammalian subject.

It is another object of the present invention to disclose the system mentioned above, wherein said modules comprise:
a. at least one measurement appliance configured to interconnect to said modules, said measurement appliance for generating a plurality of output signals indicating information related to the (i) identified predetermined characteristics of said mammalian subject microbiota; (ii) recorded data on the outcome of said in vitro contacting (iii) selected and administered cannabinoid composition (iv) response of said mammalian subject (v) processed correlating recorded in vitro data with response of said subject;
b. an analysis module operable to perform analysis of said data, said module interconnected to a processor,
c. a processor operable to execute computer program modules, said program modules comprise a genomic module, an in vitro module and a diagnosis module,
d. a memory associated with the processor,
e. a database associated with said processor and said memory,
f. a computer system comprising a processor and means for controlling the processor to carry out the method of claim 1 and a computer program executable by the processor and stored on a computer readable medium,
g. a central device further comprised a computer-readable medium storing instructions that, when executed by a computer, cause it to perform a specified method.

It is another object of the present invention to disclose the system mentioned above, wherein said mammalian subject is a human patient.

It is another object of the present invention to disclose the system mentioned above, wherein said microbiota comprised at least one of bacteria, archaea, fungi, protists, viruses, and any combination thereof.

It is another object of the present invention to disclose the system mentioned above, wherein said system additionally configured to identify characteristics of circulating tumor cells (CTCs) of said mammalian subject.

It is another object of the present invention to disclose the system mentioned above, wherein said system additionally configured to identify characteristics of cancerous tissue of said mammalian subject.

It is another object of the present invention to disclose the system mentioned above, wherein said characteristics of said mammalian subject microbiota is selected from the group of means consisting counting live cells, counting dead cells, counting apoptotic cells, measuring levels of RNA, measuring levels of DNA and any combination thereof.

It is another object of the present invention to disclose the system mentioned above, wherein said human patient selected from a group of patients not diagnosed with cancer, patients not diagnosed with microbial infections, patients diagnosed with cancer, patients diagnosed with cancer resistant to conventional chemotherapies, patients diagnosed with microbial infections and patients diagnosed with microbial infections resistant to conventional antibiotics and any combination thereof.

It is another object of the present invention to disclose the system mentioned above, wherein microbial infections comprise at least one of bacterial infections, fungal infections, viral diseases, parasite diseases, prion diseases and any combination thereof.

It is another object of the present invention to disclose the system mentioned above, wherein said microbial infection is Gonorrhea.

It is another object of the present invention to disclose the system mentioned above, wherein said microbial infection is Candida.

It is another object of the present invention to disclose the system mentioned above, wherein said response is selected of a group comprising: cardiac monitoring, hemodynamic monitoring, respiratory monitoring, neurological monitoring, blood glucose monitoring, childbirth monitoring, body temperature monitoring, vital parameters, local response monitoring and any combination thereof.

It is another object of the present invention to disclose the system mentioned above, wherein cancer is selected from gastric cancer, esophageal cancer, vaginal cancer, colorectal cancer, hepatobiliary cancer, pancreatic cancer, lung cancer, stomach cancer, head and neck cancer, breast cancer, lymphoma, cutaneous B-cells non-Hodgkin lymphoma, MALT lymphomas, Hodgkin lymphoma, colorectal adenoma and any combination thereof.

It is another object of the present invention to disclose the system mentioned above, wherein said cannabinoid is selected from the group consisting of cannabinoid-type, cannabinoid derivative, cannabis extract or fraction thereof, non-cannabinoid-type constituent, product, compound, molecule or substance and any combination thereof.

It is another object of the present invention to disclose the system mentioned above, wherein said cannabinoid compound is extracted from cannabis; said cannabis is selected from a group consisting of: Cannabis sativa, Cannabis indica, Cannabis ruderalis, and any combination thereof.

It is another object of the present invention to disclose the system mentioned above, wherein said cannabinoid compounds are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

It is another object of the present invention to disclose the system mentioned above, wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

It is another object of the present invention to disclose the system mentioned above, wherein the cannabidiol (CBD) or a derivative thereof is selected from the group consisting of CBD, CBDV, CBDA and any combination thereof.

It is another object of the present invention to disclose the system mentioned above, wherein said selecting of cannabinoid composition comprising at least one cannabinoid compound determined to have modulated said microbiota, adjunct to conventional chemotherapy; or said cannabinoid compound as mono-therapy.

It is another object of the present invention to disclose the system mentioned above, wherein said conventional chemotherapy is selected from the group consisting of chemotherapy, surgery, radiotherapy, hormonotherapy, and/or immunotherapy.

It is another object of the present invention to disclose the system mentioned above, wherein said selected cannabinoid composition comprising at least one cannabinoid compound determined to have modulated said microbiota adjunct to conventional antibiotics; therapy or said cannabinoid compound as mono-therapy.

It is another object of the present invention to disclose the system mentioned above, wherein said response is selected from the group consisting of: cancer markers level, tumor size monitoring, metastasis monitoring, survival, quality of life measured according to one or more scales, and any combination thereof.

It is another object of the present invention to disclose the system mentioned above, wherein said response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, inhibited microbial infection, inhibited microbial growth, improved mammalian subject survival, or any combination thereof.

It is another object of the present invention to disclose the system mentioned above, repeating the recording data on the outcome of in vitro contacting of said microbiota at a plurality of time points, determining whether the subject is responsive; and recommending the administration of the selected personalized therapy be continued if the subject is responsive, or to be discontinued is the subject is non responsive, i.e. resistant.

It is another object of the present invention to disclose the system mentioned above, wherein resistance to a drug is detected when there is uninhibited microbial infection, uninhibited microbial growth uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

It is another object of the present invention to disclose the system mentioned above, wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

It is another object of the present invention to disclose the system mentioned above, wherein the recorded data on the outcome of in vitro contacting comprises steps selected from a group of isolating, enumerating, counting, purifying, sequencing, sensitizing, with a plurality of cannabinoid compounds and any combination thereof.

It is another object of the present invention to disclose the system mentioned above, wherein said recording data on the outcome of in vitro contacting is selected from the group of means consisting of: optic, luminescent, fluorescent, immunological, cell count, radioactive, non-radioactive isotopic, electrical and any combination thereof.

It is another object of the present invention to disclose the system mentioned above, wherein said system further comprises recording data of said in vitro contacting microbiota with a plurality of cannabinoid compounds at n time points, wherein n is an integer equal or higher than 2, comprising of at least one time point before start of said personalized modulation and at least a second time point at a time during said first cycle; and correlating said in vitro data with said therapeutic response of said first cycle and selecting a second cycle of said personalized modulation for said mammalian subject.

It is another object of the present invention to disclose the system mentioned above, wherein said system is additionally useful for early diagnosing of cancerous states.

It is another object of the present invention to disclose the system mentioned above, wherein said system is additionally useful for early diagnosing of microbial infections.

### BRIEF DESCRIPTION OF THE FIGURES

In order to understand the invention and to see how it may be implemented in practice, a plurality of embodiments is adapted to now be described, by way of non-limiting example only, with reference to the accompanying drawings,

FIG. 1: A system for personalized cannabinoid-base modulation of microbiota for mammalian subjects.

FIG. 2: A method for personalized cannabinoid-base modulation of microbiota for mammalian subjects.

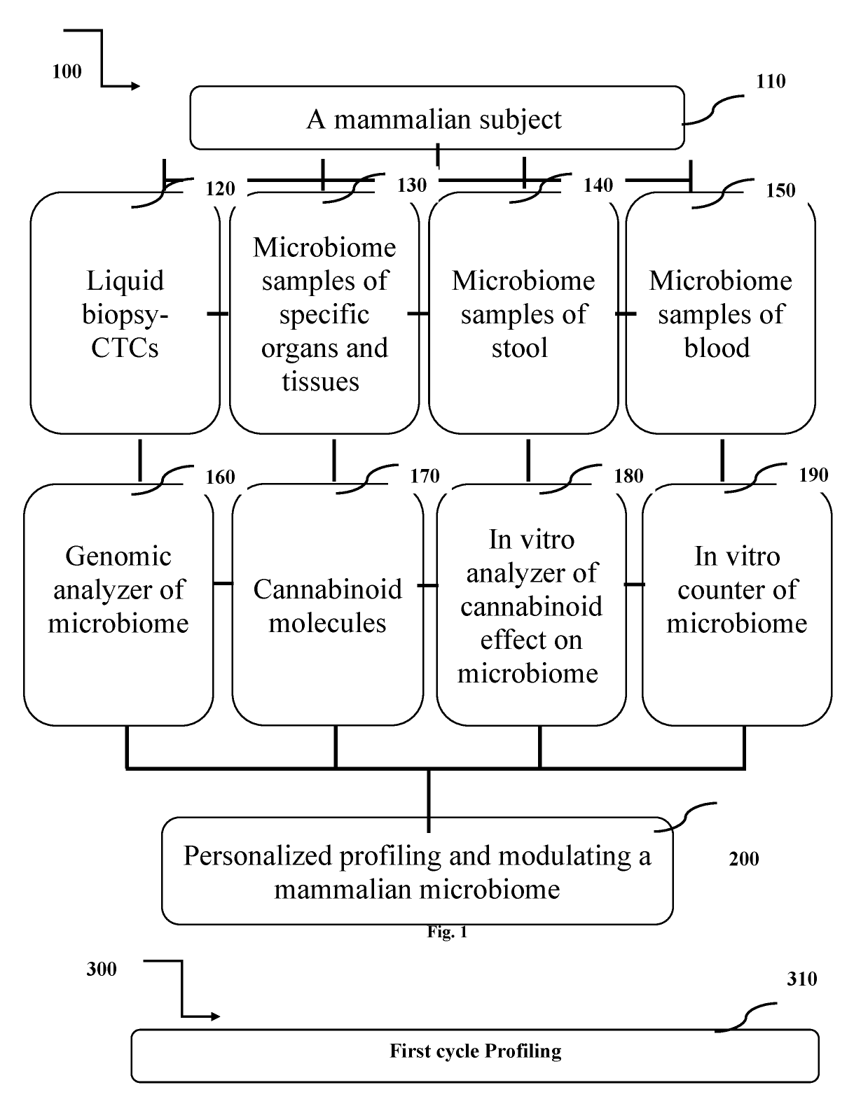

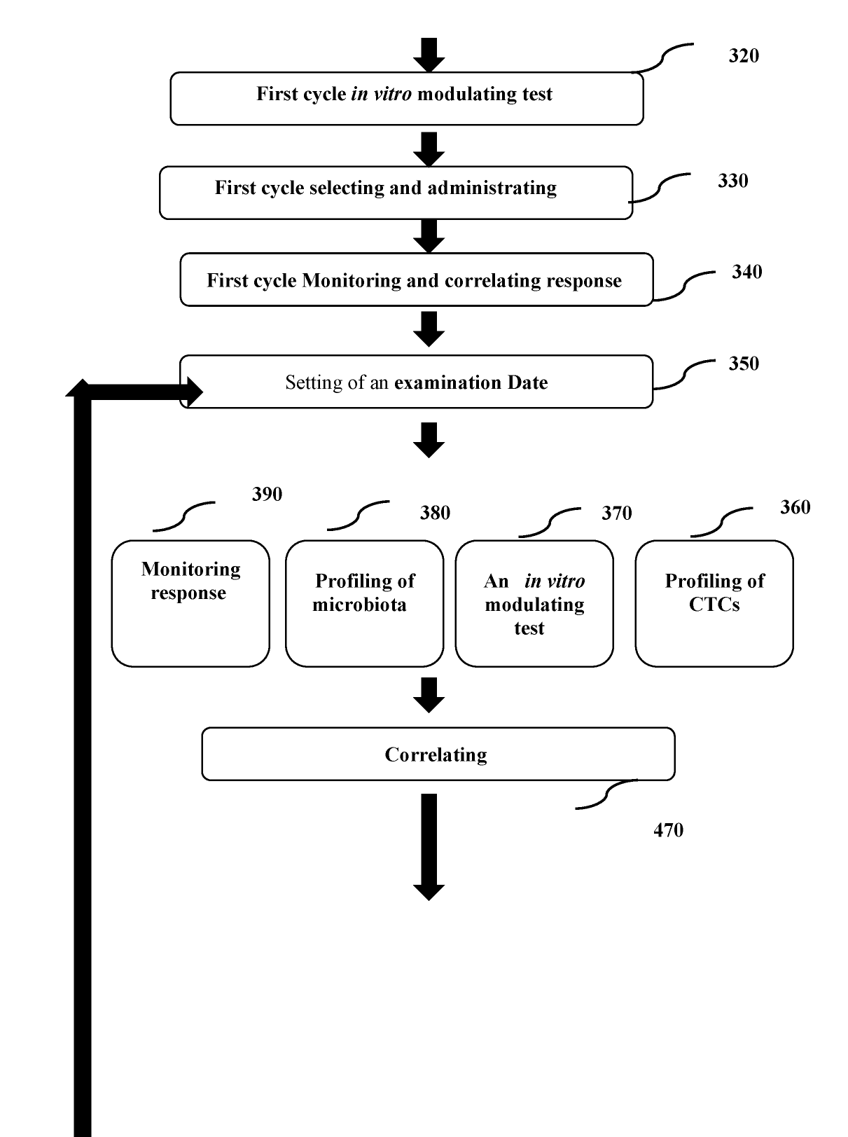

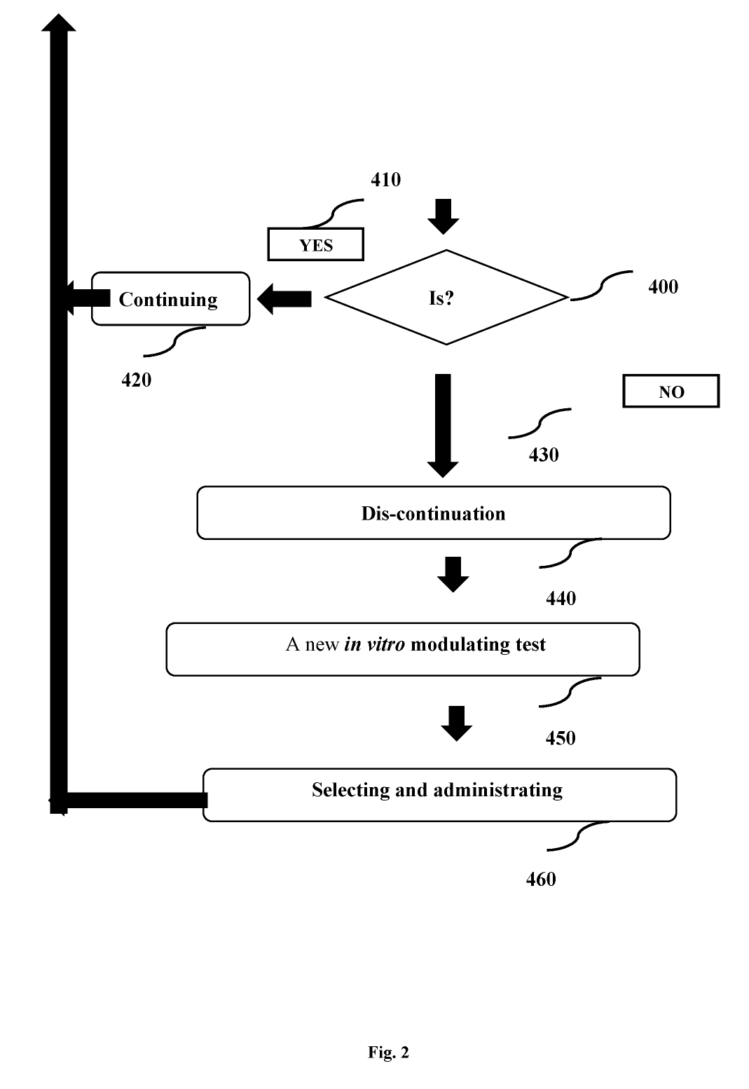

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a novel method for assessing the clinical status of a mammalian subject by profiling the individual microbiome of said subject; in vitro modulating said microbiome by with cannabis-based compounds; selecting the most relevant microbiome-modulating cannabis-based compound; and further clinically assessing of said selected compound on said mammalian subject.

Apart from the various chemical and physical (ionizing radiation, UV light) carcinogens, several biological agents can also contribute to the development of cancer; for example viruses, bacteria and parasites can potentiate a carcinogenic process in humans.

Likewise, the human microbiome, which includes the collective genome of all bacteria, archaea, fungi, protists, and viruses found in and on the human body, is altered in various disease states and may substantially affect cancer risk.

Microbiota and host form a complex ‘super-organism’ in which symbiotic relationships confer benefits to the host in many key aspects of life. However, defects in the regulatory circuits of the host that control bacterial sensing and homeostasis, or alterations of the microbiome, through environmental changes (infection, diet or lifestyle), may disturb this symbiotic relationship and promote disease. Increasing evidence indicates a key role for the bacterial microbiota in carcinogenesis.

Previously detected associations of individual bacteria (e.g., Helicobacter pylori), periodontal disease, and inflammation with specific cancers have motivated studies considering the association between the human microbiome and cancer risk. There is epidemiologic evidence for associations between the human microbiome and cancer, particularly gastric and colorectal cancer. However, epidemiologic studies of this association have thus far been very limited, typically with small sample sizes and cross-sectional designs with single-time sampling.

Microbiome associations with cancer may differ across many host factors, including sex, age, smoking, alcohol consumption, diet, obesity, physical inactivity, and polymorphisms in major malignancies, therefore explicit consideration of these host factors in a personalized approach is needed.

The present invention discloses a novel method for assessing the clinical status of a mammalian subject by profiling the individual microbiome of said subject; in vitro modulating said microbiome by with cannabis-based compounds; selecting the most relevant microbiome-modulating cannabis-based compound; and further clinically assessing of said selected compound on said mammalian subject.

The current invention discloses repeated, prospectively collected oral, fecal, nasal, gastrointestinal, vaginal, pulmonary microbiome floral and tissue sampling, as well as other samples, which will be important to indicate the temporal nature of microbial profiling, as well as associations with diseases such as cancer and infective disorders.

The microbiome analysis disclosed in the current invention includes analysis of bacteria and archaea, as well as fungi, protists, and viruses, to fully characterize the human microbiome and its relationship with malignancies' risk.

This personalized profiling of microbiota will be accompanied with cancerous tissue and Circulating Tumor Cells (CTCs) analysis of the same individual mammalian subject.

This novel invention provides opportunities to manipulate the microbiota by using personalized cannabinoid practice for improving treatment of cancerous and inflectional malignancies, and to reduce morbidity and mortality by ameliorating screening and prevention.

Furthermore, specific embodiments of the current invention, enable acceleration of the diagnosis of cancer or pre-cancerous conditions, to increase efficacy and reduce toxicity of cancer therapy and ideally to prevent cancer by interrupting a microbial carcinogenic pathway.

Other embodiments of the current invention incorporate probiotics as adjuvants the cannabis-based personalized therapy, thereby harnessing microbiota to improve cancer and infections' care.

### Microbiome

The human body is not only a complex group of organs and systems, but also contains more than 500 different species of microorganisms that accompany human from birth to death. Human biological entity is a stable symbiosis of two equal autonomous systems: macro-organism (host) and symbiotic microorganisms that are evolutionarily adapted to life in relatively open human organs on the basis of mutually beneficial relations.

These symbiotic microorganisms is called the microbiome and comprises a wide variety of microorganisms, including archaea, viruses and fungi, although anaerobic bacteria is the most studied group since they are the most abundant These microbial communities are acquired at birth and are essential for maintaining body homeostasis.

The human microbiota consists of the 10-100 trillion symbiotic microbial cells harbored by each person, primarily bacteria in the gut although there are different communities residing in a vast range of body niches. Microorganism species composition depends on the organ inhabited, and also exhibit a large inter-individual difference in microflora.

The interaction between the host and the microbiome is dynamic and controlled by a vast number of genetic and environmental factors, such as age, geography, alcohol or drug intake and diet. However, although intestinal microbiota have been shown to be individual and variable over time, only two predominant phyla, namely Firmicutes and Bacteroidetes, comprise over 90% of all endogenous bacteria present in healthy adults. Other members of the normal colonic microbiome include Eubacterium, Bifidobacterium, Fusobacterium, Lactobacilli, Enterococci, Streptococci or Enterobacteriaceae. The composition of the microbiome is mainly studied using rRNA sequences as traditional culture methods are inadequate in most cases.

Thus, due to its vast importance, alterations in normal flora cause serious complications. For instance, the condition known as dysbiosis, in which the natural relationship between the host and the intestinal microbiota is disrupted is considered to be one of the most probable causes of inflammatory bowel disease (IBD) or colorectal cancer (CRC). Many factors, such as antibiotic treatment or some types of diet, are known to be involved in the development of dysbiosis.

Furthermore, our bodies are continuously exposed to microbial cells, both resident and transient, as well as their byproducts, including toxic metabolites. Circulation of toxic metabolites may contribute to cancer onset or progression at locations distant from where a particular microbe resides. Moreover, microbes may migrate to other locations in the human body and become associated with tumor development.

### Circulating-Tumor-Cells

Circulating-Tumor-Cells (CTC) provide a blood biomarker for early carcinogenesis, cancer progression and treatment effectiveness. An increase in CTCs is associated with cancer progression, a CTC decrease with cancer containment or remission. CTCs can be useful for screening cancer drugs as they may reflect the severity and heterogeneity of primary tumors. CTCs are present in the blood of many patients with solid tumors. The clinical value of CTCs as a biomarker for early cancer detection, diagnosis, prognosis, prediction, stratification and pharmacodynamics has been widely explored in recent years. However, the clinical utility of current CTC tests is limited mainly due to methodological constraints.

A full cancer genome and transcriptome is present in each individual CTC therefore these cells represent an interesting source of tumor information where mutations and gene fusions can be found. Many studies have used enumeration of CTCs to predict recurrence or used cancer-derived biomarkers to follow therapy resistance. However, elaborate techniques are required to isolate pure CTCs as residual immune cells often contaminate the CTC population.

Assessment and enumeration of CTCs-Maintrac (SIMFO GmbH, Bayreuth, Germany)

Maintrac is a diagnostic platform based on microscopic identification of circulating tumor cells. Maintrac uses only two steps for identification, thus prevents damage and loss of the cells during the process,. In contrast to many other methods, Maintrac does not purify the cells or enriches them, but identifies them within the context of the other blood compounds. To obtain vital cells and to reduce stress of those cells, blood cells are prepared by only one centrifugation step and erythrocyte lysis. Maintrac uses an EpCAM antibody, which is used as a fluorescent marker to identify those cells and is not used for enrichment. Together with the nuclear staining with Propidium iodide, Maintrac method can distinguish between dead and living cells. Vitals cell are identified as Propidium excluding EpCAM positive cells and counted as potential tumor cells and further analyzed by fluorescence microscopy.

Unlike other methods Maintrac does not use the single cell count as a prognostic marker, rather Maintrac utilizes the dynamics of the cell count. Rising tumor cell numbers are an important factor that tumor activity is ongoing. Decreasing cell counts are a sign for a successful therapy.

Therefore, Maintrac can be used in following situations:
To verify the success of a chemotherapy
To diagnosis for cancer recurrence
To diagnosis for drug resistance
Maintrac is a method which enables detecting of a maximum of unselected non-hematological, epithelial cells in the blood, assuming that in cancer patients the majority of these cells are derived from the tumor. Assessment of the number of these cells longitudinally during the course of disease and therapy allows the response to different treatments to be monitored. Due to the viability of the cells, additional analyses such as expression profiles and determination of their sensitivity to drugs can be performed.

### Cannabinoids

Cannabinoids were primarily discovered in marijuana (cannabis flower) and hashish (compressed cannabis resin) from the plant of Cannabis sativa. This plant contains more than 80 phyto-cannabinoids. The main active constituent of marijuana is the psychoactive Δ9-tetrahydrocannabinol (Δ9-THC), which acts at cannabinoid 1 (CB1) and cannabinoid 2 (CB2) receptors as a partial agonist. Other important natural cannabinoids present in marijuana are the non-psychoactive cannabidiol (CBD), Δ9-tetrahydro-cannabivarin (Δ9-THCV) and cannabichromene (CBC) [1-3]. Among them CBD has attracted the greatest attention thus far. It was shown to antagonize the effects of CB1/CB2 receptor agonists, to counteract the psychotropic and other negative effects of Δ9-THC and several data suggest that it behaves as an inverse agonist of CB1 and CB2 receptors.

Some of these plant-derived cannabinoids are used in the medical practice, such as Δ9-THC (dronabinol) and its synthetic analogue, nabilone against chemotherapy-induced nausea and emesis, and as appetite stimulants (e.g. in AIDS patients). CBD combined with Δ9-THC (nabiximols) is used to relief neuropathic pain and spasticity in multiple sclerosis, and as an adjunctive analgesic treatment in advanced cancer pain.

Besides phyto-cannabinoids another group of naturally occurring substances that interact with cannabinoid receptors are the endo-cannabinoids. These lipid mediators are not stored but synthesized on demand in a site- and time-dependent manner and are rapidly degraded after exerting a transient and localized effect. The endo-cannabinoids include inter alia endocannabinoid, N-arachidonoylethanolamine or anandamide (AEA); 2-arachidonoylglycerol (2-AG); homo-y-linolenoylethanolamine, 7,10,13,16-docosatetraenoylethanolamide, 2-arachidonoylglycerol ether (2-AGE, noladin ether), O-arachidonoyl ethanolamine (virodhamine) and N-arachidonoyl dopamine (NADA).

### Microbiota Profiling

In several embodiments of the current invention, microbiota profiling will performed by PCR amplification of the 16S rRNA gene, followed by sequencing. In several embodiments of the current invention, microbiota profiling is performed by either growing organisms in pure culture; by small-subunit ribosomal RNA (rRNA) studies, in which the 16S rRNA gene sequences (for archaea and bacteria) or the 18S rRNA gene sequences (for eukaryotes) are used as stable phylogenetic markers to define which lineages are present in a sample, and meta-genomic studies, in which community DNA is subject to shotgun sequencing. The term ‘about’ hereinafter refers to a range of 25% below or above a quoted value.

### EXAMPLE 1

Profiling microbiota is performed personally for each mammalian subject, because of a great inter-individual difference in microflora. The simplest method is to count the bacteria number in blood, tissue and organs samples, like fecal samples, as well noninvasive samples from other organs like colon, the anterior nares (nose), posterior pharynx (throat), skin etc.

A human patient (110) is admitted to a physician clinics for first visit. At the clinics, samples of stool (140), blood (150) and selected organs (130) such as nose, throat and lungs are taken; as well as liquid biopsy of CTCs (120), are taken for microbiome profiling and modulating. The patient's microbiome samples undergo the first cycle profiling (310), comprising identifying predetermined characteristics of the microbiome in said samples. Said samples are in vitro counted by the counter (190), while the microbiome genome of these samples is in vitro analyzed by a genomic analyzer (160).

Then, a first cycle in vitro modulating test (320) is performed by in vitro contacting of at least one of said sample of microbiota with a plurality of cannabinoid compounds and recording data obtained of this modulating test. The results of said modulating test (320) serve as a basis for selecting cannabinoid composition determined to have modulated said microbiota in vitro; and administrating said cannabinoid composition to said mammalian subject (330).

The patient is further monitored for his/her response for the treatment by cardiac monitoring, hemodynamic monitoring, respiratory monitoring, neurological monitoring, blood glucose monitoring, childbirth monitoring, body temperature monitoring, vital parameters, or local response monitoring and any combination thereof. The obtained monitored response of said patient (340) is correlated with in vitro obtained data.

Then, a second examination time/date is set for said mammalian subject (350). During the new examination date, the patient's samples of samples of stool (140), blood (150) and selected organs (130); as well as liquid biopsy of CTCs (120), are taken for microbiome profiling and modulating. Several tests are performed during the second examination date:
i. Profiling of CTCs in liquid biopsy (360)
ii. In vitro cannabis-based modulating test on microbiota(370)
iii. Profiling of microbiota in patient's samples (380)
iv. Monitoring response of said mammalian (390)

The monitored response of said patient during the second examination (390) is correlated with in vitro obtained data (470).

Then the data and parameters are analyzed (400):
i. Is the number of microbiota changing?
ii. Is number of CTCs decreasing?
iii. Are the parameters measured as a patient response ameliorating (400)

If the answer is YES (410), then the present clinical personalized cannabis treatment is continued (420; and a new examination date is set (350).

If the answer is NO (430), the present cannabis treatment is discontinued. (440). Then, a new in vitro modulating test comprises of in vitro contacting of a sample of microbiota with a plurality of cannabinoid compounds and recording data (450) is performed; followed by selecting and administrating of a new personalized cannabinoid compound according to modulation test of the new plurality of the cannabis-based compounds (460).

Reference is now made to an embodiment of the present invention disclosing a method useful for personalized time determined profiling and modulating a microbiome of a mammalian subject, wherein said steps comprise:
f. identifying predetermined characteristics of said mammalian subject microbiota;
g. contacting in vitro a sample of said microbiota with a plurality of cannabinoid compounds and recording data;
h. selecting a cannabinoid composition comprising at least one cannabinoid compound determined to have modulated said microbiota in vitro and administering it to said mammalian subject;
i. monitoring response of said mammalian subject to said administration and correlating said response with said in vitro data
j. correlating said in vitro data with said response of said mammalian subject first cycle and optionally selecting a second cycle of said personalized profiling and modulation for said mammalian subject.

Reference is now made to an embodiment of the present invention disclosing the method mentioned above, wherein mammalian subject said is a human patient.

Reference is now made to an embodiment of the present invention disclosing the method mentioned above, wherein said microbiota comprising at least one of bacteria, archaea, fungi, protists, viruses, and any combination thereof.

Reference is now made to an embodiment of the present invention disclosing the method mentioned above, wherein said method additionally comprising steps of identifying characteristics of circulating tumor cells (CTCs) of said mammalian subject.

Reference is now made to an embodiment of the present invention disclosing the method mentioned above, wherein said method additionally comprising steps of identifying characteristics of cancerous tissue of said mammalian subject.

Reference is now made to an embodiment of the present invention disclosing the method mentioned above, wherein said identifying characteristics of said mammalian subject microbiota is selected from the group of means consisting counting live cells, counting dead cells, counting apoptotic cells, measuring levels of RNA, measuring levels of DNA and any combination thereof.

Reference is now made to an embodiment of the present invention disclosing the method mentioned above, wherein said human patient selected from a group of patients not diagnosed with cancer, patients not diagnosed with microbial infections, patients diagnosed with cancer, patients diagnosed with cancer resistant to conventional chemotherapies, patients diagnosed with microbial infections and patients diagnosed with microbial infections resistant to conventional antibiotics and any combination thereof.

Reference is now made to an embodiment of the present invention disclosing the method mentioned above, wherein microbial infections comprising at least one of bacterial infections, fungal infections, viral diseases, parasite diseases, prion diseases and any combination thereof.

Reference is now made to an embodiment of the present invention disclosing the method mentioned above, wherein said microbial infection is Gonorrhea.

Reference is now made to an embodiment of the present invention disclosing the method mentioned above, wherein said microbial infection is Candida.

Reference is now made to an embodiment of the present invention disclosing the method mentioned above, wherein said response is selected of a group comprising: cardiac monitoring, hemodynamic monitoring, respiratory monitoring, neurological monitoring, blood glucose monitoring, childbirth monitoring, body temperature monitoring, vital parameters, local response monitoring and any combination thereof.

Reference is now made to an embodiment of the present invention disclosing the method mentioned above, wherein cancer is selected from gastric cancer, esophageal cancer, vaginal cancer, colorectal cancer, hepatobiliary cancer, pancreatic cancer, lung cancer, stomach cancer, head and neck cancer, breast cancer, lymphoma, cutaneous B-cells non-Hodgkin lymphoma, MALT lymphomas, Hodgkin lymphoma, colorectal adenoma and any combination thereof.

Reference is now made to an embodiment of the present invention disclosing the method mentioned above, wherein said cannabinoid is selected from the group consisting of cannabinoid-type, cannabinoid derivative, cannabis extract or fraction thereof, non-cannabinoid-type constituent, product, compound, molecule or substance and any combination thereof.

Reference is now made to an embodiment of the present invention disclosing the method mentioned above, wherein said cannabinoid compound is extracted from cannabis; said cannabis is selected from a group consisting of: Cannabis sativa, Cannabis indica, Cannabis ruderalis, and any combination thereof.

Reference is now made to an embodiment of the present invention disclosing the method mentioned above, wherein said cannabinoid compounds are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

Reference is now made to an embodiment of the present invention disclosing the method mentioned above, wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

Reference is now made to an embodiment of the present invention disclosing the method mentioned above, wherein the cannabidiol (CBD) or a derivative thereof is selected from the group consisting of CBD, CBDV, CBDA and any combination thereof.

Reference is now made to an embodiment of the present invention disclosing the method mentioned above, wherein said selecting of cannabinoid composition comprising at least one cannabinoid compound determined to have modulated said microbiota, adjunct to conventional chemotherapy; or said cannabinoid compound as mono-therapy.

Reference is now made to an embodiment of the present invention disclosing the method mentioned above, wherein said conventional chemotherapy is selected from the group consisting of chemotherapy, surgery, radiotherapy, hormonotherapy, and/or immunotherapy.

Reference is now made to an embodiment of the present invention disclosing the method mentioned above, wherein said selecting of cannabinoid composition comprising at least one cannabinoid compound determined to have modulated said microbiota adjunct to conventional antibiotics; therapy or said cannabinoid compound as mono-therapy.

Reference is now made to an embodiment of the present invention disclosing the method mentioned above, wherein said response is selected from the group consisting of: cancer markers level, tumor size monitoring, metastasis monitoring, survival, quality of life measured according to one or more scales, and any combination thereof.

Reference is now made to an embodiment of the present invention disclosing the method mentioned above, wherein said response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, inhibited microbial infection, inhibited microbial growth, improved mammalian subject survival, or any combination thereof. Reference is now made to an embodiment of the present invention disclosing the method mentioned above, repeating the recording data on the outcome of in vitro contacting of said microbiota at a plurality of time points, determining whether the subject is responsive; and recommending the administration of the selected personalized therapy be continued if the subject is responsive, or to be discontinued is the subject is non responsive, i.e. resistant.

Reference is now made to an embodiment of the present invention disclosing the method mentioned above, wherein resistance to a drug is detected when there is uninhibited microbial infection, uninhibited microbial growth uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

Reference is now made to an embodiment of the present invention disclosing the method mentioned above, wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

Reference is now made to an embodiment of the present invention disclosing the method mentioned above, wherein the recording data on the outcome of in vitro contacting comprises steps selected from a group of isolating, enumerating, counting, purifying, sequencing, sensitizing, with a plurality of cannabinoid compounds and any combination thereof.

Reference is now made to an embodiment of the present invention disclosing the method mentioned above, wherein said recording data on the outcome of in vitro contacting is selected from the group of means consisting of: optic, luminescent, fluorescent, immunological, cell count, radioactive, non-radioactive isotopic, electrical and any combination thereof.

Reference is now made to an embodiment of the present invention disclosing the method mentioned above, wherein said method further comprising recording data of said in vitro contacting microbiota with a plurality of cannabinoid compounds at n time points, wherein n is an integer equal or higher than 2, comprising of at least one time point before start of said personalized modulation and at least a second time point at a time during said first cycle; and correlating said in vitro data with said therapeutic response of said first cycle and selecting a second cycle of said personalized modulation for said mammalian subject.

Reference is now made to an embodiment of the present invention disclosing the method mentioned above, wherein said method is additionally useful for early diagnosing of cancerous states.

Reference is now made to an embodiment of the present invention disclosing the method mentioned above, wherein said method is additionally useful for early diagnosing of microbial infections.

Reference is now made to an embodiment of the present invention disclosing a system useful for a personalized time-determined profiling and modulating a microbiome of mammalian subject, wherein the system comprises:
h. in vitro identifier module configured to identify pre-determined characteristics of said mammalian subject microbiota;
i. in vitro contact module configured to contact a sample of said microbiota with a plurality of cannabinoid compounds;
j. a recorder module for recording data on the outcome of said in vitro contacting
k. a module for selecting a cannabinoid composition comprising at least one cannabinoid compound determined to have modulated said microbiota for said mammalian subject and for administrating to said mammalian subject;
l. a module for monitoring response of said mammalian subject to said administration;
m. a module for correlating said in vitro data with said response of said mammalian subject first cycle and optionally selecting a second cycle of said personalized profiling and modulation of said mammalian subject.
n. a module for processing said detected signals with said therapeutic response of said first cycle and selecting a second cycle of clinical personalized therapy for said mammalian subject.

Reference is now made to an embodiment of the present invention disclosing the system mentioned above, wherein said modules comprise:
h. at least one measurement appliance configured to interconnect to said modules, said measurement appliance for generating a plurality of output signals indicating information related to the (i) identified predetermined characteristics of said mammalian subject microbiota; (ii) recorded data on the outcome of said in vitro contacting (iii) selected and administered cannabinoid composition (iv) response of said mammalian subject (v) processed correlating recorded in vitro data with response of said subject;
i. an analysis module operable to perform analysis of said data, said module interconnected to a processor,
j. a processor operable to execute computer program modules, said program modules comprise a genomic module, an in vitro module and a diagnosis module,
k. a memory associated with the processor,
l. a database associated with said processor and said memory,
m. a computer system comprising a processor and means for controlling the processor to carry out the method of claim 1 and a computer program executable by the processor and stored on a computer readable medium,
n. a central device further comprised a computer-readable medium storing instructions that, when executed by a computer, cause it to perform a specified method.

Reference is now made to an embodiment of the present invention disclosing the system mentioned above, wherein said mammalian subject is a human patient.

Reference is now made to an embodiment of the present invention disclosing the system mentioned above, wherein said microbiota comprised at least one of bacteria, archaea, fungi, protists, viruses, and any combination thereof.

Reference is now made to an embodiment of the present invention disclosing the system mentioned above, wherein said system additionally configured to identify characteristics of circulating tumor cells (CTCs) of said mammalian subject.

Reference is now made to an embodiment of the present invention disclosing the system mentioned above, wherein said system additionally configured to identify characteristics of cancerous tissue of said mammalian subject.

Reference is now made to an embodiment of the present invention disclosing the system mentioned above, wherein said characteristics of said mammalian subject microbiota is selected from the group of means consisting counting live cells, counting dead cells, counting apoptotic cells, measuring levels of RNA, measuring levels of DNA and any combination thereof.

Reference is now made to an embodiment of the present invention disclosing the system mentioned above, wherein said human patient selected from a group of patients not diagnosed with cancer, patients not diagnosed with microbial infections, patients diagnosed with cancer, patients diagnosed with cancer resistant to conventional chemotherapies, patients diagnosed with microbial infections and patients diagnosed with microbial infections resistant to conventional antibiotics and any combination thereof.

Reference is now made to an embodiment of the present invention disclosing the system mentioned above, wherein microbial infections comprise at least one of bacterial infections, fungal infections, viral diseases, parasite diseases, prion diseases and any combination thereof.

Reference is now made to an embodiment of the present invention disclosing the system mentioned above, wherein said microbial infection is Gonorrhea.

Reference is now made to an embodiment of the present invention disclosing the system mentioned above, wherein said microbial infection is Candida.

Reference is now made to an embodiment of the present invention disclosing the system mentioned above, wherein said response is selected of a group comprising: cardiac monitoring, hemodynamic monitoring, respiratory monitoring, neurological monitoring, blood glucose monitoring, childbirth monitoring, body temperature monitoring, vital parameters, local response monitoring and any combination thereof.

Reference is now made to an embodiment of the present invention disclosing the system mentioned above, wherein cancer is selected from gastric cancer, esophageal cancer, vaginal cancer, colorectal cancer, hepatobiliary cancer, pancreatic cancer, lung cancer, stomach cancer, head and neck cancer, breast cancer, lymphoma, cutaneous B-cells non-Hodgkin lymphoma, MALT lymphomas, Hodgkin lymphoma, colorectal adenoma and any combination thereof.

Reference is now made to an embodiment of the present invention disclosing the system mentioned above, wherein said cannabinoid is selected from the group consisting of cannabinoid-type, cannabinoid derivative, cannabis extract or fraction thereof, non-cannabinoid-type constituent, product, compound, molecule or substance and any combination thereof.

Reference is now made to an embodiment of the present invention disclosing the system mentioned above, wherein said cannabinoid compound is extracted from cannabis; said cannabis is selected from a group consisting of: Cannabis sativa, Cannabis indica, Cannabis ruderalis, and any combination thereof.

Reference is now made to an embodiment of the present invention disclosing the system mentioned above, wherein said cannabinoid compounds are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

Reference is now made to an embodiment of the present invention disclosing the system mentioned above, wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

Reference is now made to an embodiment of the present invention disclosing the system mentioned above, wherein the cannabidiol (CBD) or a derivative thereof is selected from the group consisting of CBD, CBDV, CBDA and any combination thereof.

Reference is now made to an embodiment of the present invention disclosing the system mentioned above, wherein said selecting of cannabinoid composition comprising at least one cannabinoid compound determined to have modulated said microbiota, adjunct to conventional chemotherapy; or said cannabinoid compound as mono-therapy.

Reference is now made to an embodiment of the present invention disclosing the system mentioned above, wherein said conventional chemotherapy is selected from the group consisting of chemotherapy, surgery, radiotherapy, hormonotherapy, and/or immunotherapy.

Reference is now made to an embodiment of the present invention disclosing the system mentioned above, wherein said selected cannabinoid composition comprising at least one cannabinoid compound determined to have modulated said microbiota adjunct to conventional antibiotics; therapy or said cannabinoid compound as mono-therapy.

Reference is now made to an embodiment of the present invention disclosing the system mentioned above, wherein said response is selected from the group consisting of: cancer markers level, tumor size monitoring, metastasis monitoring, survival, quality of life measured according to one or more scales, and any combination thereof.

Reference is now made to an embodiment of the present invention disclosing the system mentioned above, wherein said response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, inhibited microbial infection, inhibited microbial growth, improved mammalian subject survival, or any combination thereof. Reference is now made to an embodiment of the present invention disclosing the system mentioned above, repeating the recording data on the outcome of in vitro contacting of said microbiota at a plurality of time points, determining whether the subject is responsive; and recommending the administration of the selected personalized therapy be continued if the subject is responsive, or to be discontinued is the subject is non responsive, i.e. resistant.

Reference is now made to an embodiment of the present invention disclosing the system mentioned above, wherein resistance to a drug is detected when there is uninhibited microbial infection, uninhibited microbial growth uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof. Reference is now made to an embodiment of the present invention disclosing the system mentioned above, wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

Reference is now made to an embodiment of the present invention disclosing the system mentioned above, wherein the recorded data on the outcome of in vitro contacting comprises steps selected from a group of isolating, enumerating, counting, purifying, sequencing, sensitizing, with a plurality of cannabinoid compounds and any combination thereof.

Reference is now made to an embodiment of the present invention disclosing the system mentioned above, wherein said recording data on the outcome of in vitro contacting is selected from the group of means consisting of: optic, luminescent, fluorescent, immunological, cell count, radioactive, non-radioactive isotopic, electrical and any combination thereof.

Reference is now made to an embodiment of the present invention disclosing the system mentioned above, wherein said system further comprises recording data of said in vitro contacting microbiota with a plurality of cannabinoid compounds at n time points, wherein n is an integer equal or higher than 2, comprising of at least one time point before start of said personalized modulation and at least a second time point at a time during said first cycle; and correlating said in vitro data with said therapeutic response of said first cycle and selecting a second cycle of said personalized modulation for said mammalian subject.

Reference is now made to an embodiment of the present invention disclosing the system mentioned above, wherein said system is additionally useful for early diagnosing of cancerous states.

Reference is now made to an embodiment of the present invention disclosing the system mentioned above, wherein said system is additionally useful for early diagnosing of microbial infections.

1. A method useful for personalized time determined profiling and modulating a microbiome of a mammalian subject, wherein said steps comprise:
a. identifying predetermined characteristics of said mammalian subject microbiota;
b. contacting in vitro a sample of said microbiota with a plurality of cannabinoid compounds and recording data;
c. selecting a cannabinoid composition comprising at least one cannabinoid compound determined to have modulated said microbiota in vitro and administering it to said mammalian subject;
d. monitoring response of said mammalian subject to said administration and correlating said response with said in vitro data; and
e. correlating said in vitro data with said response of said mammalian subject first cycle and optionally selecting a second cycle of said personalized profiling and modulation for said mammalian subject.

2. The method of claim 1, wherein said mammalian subject is a human patient.

3. The method of claim 1, wherein said microbiota comprising at least one of bacteria, archaea, fungi, protists, viruses, and any combination thereof.

4. The method of claim 1, wherein said method additionally comprising steps of identifying characteristics of circulating tumor cells (CTCs) of said mammalian subject.

5. The method of claim 1, wherein said method additionally comprising steps of identifying characteristics of cancerous tissue of said mammalian subject.

6. The method of claim 1, wherein said identifying characteristics of said mammalian subject microbiota is selected from the group of means consisting counting live cells, counting dead cells, counting apoptotic cells, measuring levels of RNA, measuring levels of DNA and any combination thereof.

7. The method according to claim 2, wherein said human patient selected from a group of patients not diagnosed with cancer, patients not diagnosed with microbial infections, patients diagnosed with cancer, patients diagnosed with cancer resistant to conventional chemotherapies, patients diagnosed with microbial infections and patients diagnosed with microbial infections resistant to conventional antibiotics and any combination thereof.

8. The method of claim 7, wherein microbial infections comprising at least one of bacterial infections, fungal infections, viral diseases, parasite diseases, prion diseases and any combination thereof.

9. The method according to claim 7, wherein said microbial infection is Gonorrhea.

10. The method according to claim 7, wherein said microbial infection is Candida.

11. The method of claim 1, wherein said response is selected of a group comprising:
cardiac monitoring, hemodynamic monitoring, respiratory monitoring, neurological monitoring, blood glucose monitoring, childbirth monitoring, body temperature monitoring, vital parameters, local response monitoring and any combination thereof.

12. The method of claim 7 wherein cancer is selected from gastric cancer, esophageal cancer, vaginal cancer, colorectal cancer, hepatobiliary cancer, pancreatic cancer, lung cancer, stomach cancer, head and neck cancer, breast cancer, lymphoma, cutaneous B-cells non-Hodgkin lymphoma, MALT lymphomas, Hodgkin lymphoma, colorectal adenoma and any combination thereof.

13. The method according to claim 1, wherein said cannabinoid is selected from the group consisting of cannabinoid-type, cannabinoid derivative, cannabis extract or fraction thereof, non-cannabinoid-type constituent, product, compound, molecule or substance and any combination thereof.

14. The method according to claim 1, wherein said cannabinoid compound is extracted from cannabis; said cannabis is selected from a group consisting of: Cannabis sativa, Cannabis indica, Cannabis ruderalis, and any combination thereof.

15. The method according to claim 1, wherein said cannabinoid compounds are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

16. The method according to claim 15, wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

17. The method according to claim 15, wherein the cannabidiol (CBD) or a derivative thereof is selected from the group consisting of CBD, CBDV, CBDA and any combination thereof.

18. The method according to claim 1, wherein said selecting of cannabinoid composition comprising at least one cannabinoid compound determined to have modulated said microbiota, adjunct to conventional chemotherapy; or said cannabinoid compound as mono-therapy.

19. The method according to claim 18 wherein said conventional chemotherapy is selected from the group consisting of chemotherapy, surgery, radiotherapy, hormonotherapy, and/or immunotherapy.

20. The method according to claim 1, wherein said selecting of cannabinoid composition comprising at least one cannabinoid compound determined to have modulated said microbiota adjunct to conventional antibiotics; therapy or said cannabinoid compound as mono-therapy.

21. The method of claim 1, wherein said response is selected from the group consisting of: cancer markers level, tumor size monitoring, metastasis monitoring, survival, quality of life measured according to one or more scales, and any combination thereof.

22. The method of claim 1, wherein said response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, inhibited microbial infection, inhibited microbial growth, improved mammalian subject survival, or any combination thereof.

23. The method according to claim 1, repeating the recording data on the outcome of in vitro contacting of said microbiota at plurality of time points, determining whether the subject is responsive; and recommending the administration of the selected personalized therapy be continued if the subject is responsive, or to be discontinued is the subject is non responsive, i.e. resistant.

24. The method of claim 23, wherein resistance to a drug is detected when there is uninhibited microbial infection, uninhibited microbial growth uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

25. The method of claim 1 wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

26. The method of claim 1 wherein the recording data on the outcome of in vitro contacting comprises steps selected from a group of isolating, enumerating, counting, purifying, sequencing, sensitizing, with a plurality of cannabinoid compounds and any combination thereof.

27. The method according to claim 1, wherein said recording data on the outcome of in vitro contacting is selected from the group of means consisting of: optic, luminescent, fluorescent, immunological, cell count, radioactive, non-radioactive isotopic, electrical and any combination thereof

28. The method according to claim 1, wherein said method further comprising recording data of said in vitro contacting microbiota with a plurality of cannabinoid compounds at n time points, wherein n is an integer equal or higher than 2, comprising of at least one time point before start of said personalized modulation and at least a second time point at a time during said first cycle; and
correlating said in vitro data with said therapeutic response of said first cycle and selecting a second cycle of said personalized modulation for said mammalian subject.

29. A system useful for a personalized time-determined profiling and modulating a microbiome of a mammalian subject, wherein the system comprises:
a. in vitro identifier module configured to identify pre-determined characteristics of said mammalian subject microbiota;
b. in vitro contact module configured to contact a sample of said microbiota with a plurality of cannabinoid compounds;
c. a recorder module for recording data on the outcome of said in vitro contacting;
d. a module for selecting a cannabinoid composition comprising at least one cannabinoid compound determined to have modulated said microbiota for said mammalian subject and for administrating to said mammalian subject;
e. a module for monitoring response of said mammalian subject to said administration;
f. a module for correlating said in vitro data with said response of said mammalian subject first cycle and optionally selecting a second cycle of said personalized profiling and modulation of said mammalian subject.
g. a module for processing said detected signals with said therapeutic response of said first cycle and selecting a second cycle of clinical personalized therapy for said mammalian subject,
further wherein said modules comprise:
at least one measurement appliance configured to interconnect to said modules, said measurement appliance for generating a plurality of output signals indicating information related to the (i) identified predetermined characteristics of said mammalian subject microbiota; (ii) recorded data on the outcome of said in vitro contacting (iii) selected and administered cannabinoid composition (iv) response of said mammalian subject (v) processed correlating recorded in vitro data with response of said subject;
an analysis module operable to perform analysis of said data, said module interconnected to
a processor,
said processor operable to execute computer program modules, said program modules comprise a genomic module, an in vitro module and a diagnosis module,
a memory associated with the processor,
a database associated with said processor and said memory,
a computer system comprising a processor and means for controlling the processor to carry out the method of claim 1 and a computer program executable by the processor and stored on a computer readable medium,
a central device further comprised a computer-readable medium storing instructions that, when executed by a computer, cause it to perform a specified method.
